(19) United States
(12) Patent Application Publication
(10) Pub. No.: US 2019/0151297 A1
(43) Pub. Date: May 23, 2019

(54) Veterinary Composition

(71) Applicant: Alan THOMPSON, Doncaster (AU)

(72) Inventor: Alan THOMPSON, Doncaster (AU)

(73) Assignee: Nauts Equine Brand Pty Ltd, Doncaster (AU)

(21) Appl. No.: 16/097,036

(22) PCT Filed: Aug. 23, 2016
(86) PCT No.: PCT/AU2016/050773

§ 371 Date: Oct. 26, 2018

(30) Foreign Application Priority Data
Apr. 29, 2016 (AU) 2016901584

(51) Int. Cl. A61K 31/4439; A61K 9/00; A61P 1/04; A61K 47/44

(52) U.S. Cl. CPC A61K 31/4439; A61K 9/0056; A61K 33/08; A61K 47/44; A61P 1/04

(57) ABSTRACT

The present invention relates to a veterinary composition for oral administration, said veterinary composition including an antacid and a therapeutically effective amount of a proton pump inhibitor, wherein the proton pump inhibitor is provided in an enteric coated form.

### FIELD OF THE INVENTION

The present invention relates to veterinary compositions, particularly veterinary compositions for oral administration including a proton pump inhibitor and an antacid, and associated products and methods.

### BACKGROUND OF THE INVENTION

A gastric or duodenal ulcer is a defect in the stomach lining, usually a break or lesion. In animals, the underlying cause of gastric ulcers is often attributed to stress events. For example, stomach acidity may be caused by the secretion of gastric acid by parietal cells. The acidity of the stomach is often buffered by alkaline saliva and mucous. Disruption or change to alkaline saliva and mucous production, for example by a stress event, may result in increased stomach acidity. It may also result in abdominal cavity pressure which may bring acidic gastric juices into contact with the sensitive upper reflux area of the stomach. Increased stomach acidity and/or abdominal cavity pressure is thought to lead to or contribute to gastric or duodenal ulcers.

An enzyme system involved in stomach acid production is the H+/K+ ATPase enzyme system, or proton pump, at the secretory membrane of parietal cells. The proton pump catalyses the exchange of hydrogen ions for potassium ions in the final stage of hydrochloric acid production. Inhibition of this enzyme system by proton pump inhibitors decreases gastric acid production.

Certain proton pump inhibitors are known, including the benzimidazole compounds omeprazole, lansoprazole, pantoprazole and leminoprazole. For example, there are products containing omeprazole currently registered in Australia for treating or preventing gastric ulcers in horses. However, these products suffer from one or more of a number of deficiencies associated with ineffectiveness, high dosage amounts and poor product consistency leading to difficulties in administration. There is therefore a need for an improved means of treating gastric ulcers in animals, particularly horses.

It is an aim of the present invention to alleviate one or more of the deficiencies associated with the prior art.

### SUMMARY OF THE INVENTION

In one aspect, the present invention provides a veterinary composition for oral administration, said veterinary composition including an antacid and a therapeutically effective amount of a proton pump inhibitor, wherein the proton pump inhibitor is provided in an enteric coated form.

By a “therapeutically effective amount” is meant an amount that elicits a biological or medicinal response when administered to an animal.

The selection of the proton pump inhibitor is not particularly limited. For example, the proton pump inhibitor may be a benzimidazole compound or combination thereof. Representative benzimidazole compounds include, but are not limited to, omeprazole, lansoprazole, pantoprazole and rabeprazole, with chemical structures as follows.

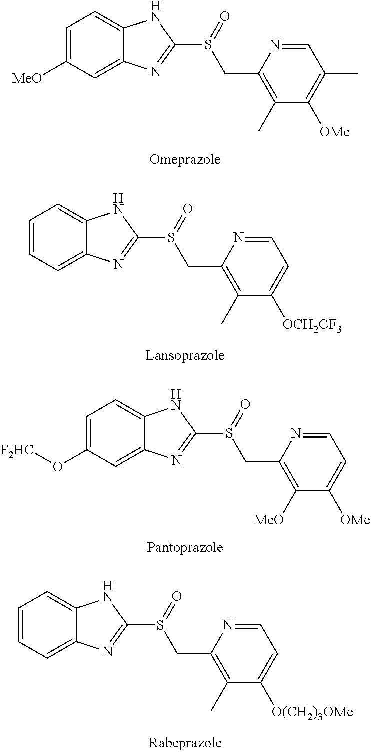

Many proton pump inhibitors, including benzimidazoles, may be present in a number of chemical forms, including for example (R)- and (S)-stereoisomers, pharmaceutically acceptable salts, polymorphs and mixtures thereof. Thus, as used herein, general or specific reference to a proton pump inhibitor includes any one or more of its chemical forms; e.g., the term ‘omeprazole’ includes stereoisomers (e.g. esomeprazole), salts, polymorphs and mixtures thereof.

In preferred embodiments, the proton pump inhibitor includes omeprazole. Omeprazole, or 5-methoxy-2[(4-methoxy-3,5-dimethylpyridin-2-yl)methylsulphinyl]-1H-benzimidazole, is a potent proton pump inhibitor disclosed in U.S. Pat. No. 4,255,432.

Many proton pump inhibitors, including the benzimidazoles and particularly omeprazole, are acid-labile and susceptible to breakdown in the acidic environment of the stomach. Without wishing to be limited by theory, by providing the proton pump inhibitor in an enteric coated form in a veterinary composition of the present invention, acid-labile proton pump inhibitors are protected from acid breakdown in the stomach and released in the less acidic or alkaline regions of the gastrointestinal tract. The proton pump inhibitor may then be absorbed and delivered to the active site of parietal cells via the blood stream. A higher proportion of the proton pump inhibitor may thus reach the active site compared with acid-labile proton pump inhibitors which are not provided in an enteric coated form, and consequently a lower dose of proton pump inhibitor may be administered for an equal therapeutic effect.

The enteric coated form in which the proton pump inhibitor may be provided includes, for example, pellets, particles, granules, beads and tablets. The proton pump inhibitor may be provided in the enteric coated form in any suitable amount. This may be, for example, an amount of about 1% to about 95%, preferably from about 5% to about 50%, and more preferably from about 8% to about 30%, by weight of the enteric coated form. The enteric coated form may be present in the veterinary composition in any suitable amount, for example an amount of about 5% to about 60%, preferably about 10% to about 50%, or more preferably about 20% to about 40%, by weight of the veterinary composition. Accordingly, the proton pump inhibitor may be present in the veterinary composition in an amount of, for example, about 0.05% to about 55% by weight of the veterinary composition, preferably about 0.5% to about 25% by weight of the veterinary composition, or more preferably about 2% to about 10% by weight of the veterinary composition.

In preferred embodiments, the proton pump inhibitor is provided in the form of enteric coated pellets. Enteric coated pellets preferably include at least a core containing the proton pump inhibitor, and an enteric coating. The enteric coating may include, for example, one or more substances selected from the group consisting of cellulose acetate phthalate, hydroxypropyl methyl cellulose phthalate, shellac, methacrylic acid copolymer and/or any other substances suitable for use in an enteric coat. Preferably, the enteric coating includes a methacrylic acid copolymer such as poly(methacrylic acid-co-ethyl acrylate). The core may also include an inert carrier substance. The inert carrier substance may include, for example, sugar spheres including lactose or mannitol, starch, cellulose or nonpareil seeds and/or any other suitable substance.

The enteric coated pellets may also include a sealing layer between the core and the enteric coating. The sealing layer may include, for example, one or more substances selected from the group consisting of polyvinylpyrrolidone, methylcellulose, hydroxymethylcellulose, hydroxypropylmethylcellulose, polyethylene glycol, polyvinyl alcohol and/or any other suitable substance. Preferably the sealing layer is water-soluble and includes hydroxypropylmethylcellulose.

Other suitable substances for inclusion in the pellets may be one or more of colourants, buffers, surfactants, binders, sweeteners, preservatives, plasticisers, glidants and opacifiers, for example. Representative suitable enteric coated pellets include those as described in, for example, WO2006/026829.

The enteric coated pellets may be any size fit for purpose. For example, the pellets may be of a size such that not less than 90% pass through an ASTM 20 mesh sieve, preferably a 16 mesh sieve and most preferably a 12 mesh sieve. Alternatively or in addition, the pellets may be of a size such that not more than 10% pass through an ASTM 24 mesh sieve, preferably a 20 mesh sieve and most preferably a 16 mesh sieve.

The antacid may be any substance which acts to at least partially neutralise the acidity of the stomach. This may include, for example, a magnesium, calcium, sodium and/or aluminium salt. Representative examples include any one or more of magnesium oxide, magnesium hydroxide, calcium carbonate, sodium bicarbonate, magnesium aluminate monohydrate, aluminium hydroxide, magnesium trisilicate, aluminium carbonate, magnesium carbonate, aluminium magnesium hydroxide sulfate and sodium citrate.

Preferably, the antacid is magnesium oxide, and particularly preferably magnesium oxide light.

The combination of the antacid and the proton pump inhibitor in an enteric coated form gives rise to certain benefits. It has been found that the combined effect of the antacid and the proton pump inhibitor when delivered in an enteric coated form, provides a particularly effective veterinary composition for the treatment or prevention of a gastric ulcer condition in an animal. In particular, magnesium oxide has been found to be a surprisingly effective antacid when used in a veterinary composition of the present invention. Magnesium oxide is also readily absorbed in animals, particularly horses, such that magnesium becomes biologically available. This gives rise to several associated subsidiary benefits. For example, magnesium is an essential mineral in animals. It is involved in many biological functions including in bone integrity, as an enzyme co-factor and protection against inflammation and damage by endotoxins. Magnesium is also involved in muscle relaxation and nerve excitability and is a calming mineral. Further, gastric ulcers often cause discomfort, loss of appetite and general irritability in animals. Improved temperament, appetite, coat shine and general well-being of an animal is attributed to the use of magnesium, and particularly magnesium oxide, in a veterinary composition of the present invention.

Thus, in preferred embodiments, the antacid is not enteric coated, such that it may be released in the stomach. Preferably, the antacid may be present in the veterinary composition in an amount of about 5% to about 25%, more preferably about 10% to about 20%, by weight of the veterinary composition. In preferred embodiments, the antacid is present in an amount of about 12% to about 16% by weight of the veterinary composition.

The veterinary composition of the present invention may be formulated in any dosage form suitable for oral delivery. This may include, for example, a paste, solution, suspension, gel, powder, granule, tablet or capsule. The veterinary composition of the present invention may be formulated with any suitable excipients. Suitable excipients may be selected based on the desired dosage form and animal to which administration is intended, among other factors. A formulator skilled in the art may readily determine suitable excipients. For example, suitable excipients for a paste dosage form may include carriers, thickening agents, preservatives, flavours, colours, sweeteners and/or antioxidants, among others. Suitable preservatives may include parabens; flavours may include caramel, carrot and/or apple; colours may include iron oxide and/or titanium dioxide; sweeteners may include sugars; and antioxidants may include butylated hydroxytoluene (BHT) and/or butylated hydroxyanisole (BHA).

In preferred embodiments, the veterinary composition is formulated in a paste dosage form. A paste dosage form has the advantage of reduced loss by dripping or expulsion from the mouth during administration, and better dosage accuracy. Further, the paste may be packaged in a dial-dose tube for ease of administration and/or individualised dosage administration.

In preferred embodiments, the veterinary composition is formulated with a carrier and/or a thickening agent. In particularly preferred embodiments, the veterinary composition includes a carrier and a thickening agent, and no other excipients.

The carrier may be any carrier fit for purpose. For example, in preferred embodiments the carrier may be an oil. This may include an oil selected from the group consisting of one or more of mineral oil, avocado oil, castor oil, cottonseed oil, ethyl oleate, isopropyl myristate, isopropyl palmitate, myristyl alcohol, octyldodecanol, peanut oil, sunflower oil, capric triglycerides, caprylic triglycerides, safflower oil, sesame oil, canola oil, aniseed oil, macadamia nut oil, olive oil, squalene, rice bran oil, soya oil and almond oil. In particularly preferred embodiments, the carrier includes canola oil.

The carrier may be present in the veterinary composition in any amount fit for purpose. This may include, for example, an amount of about 30% to about 85%, more preferably about 40% to about 70%, by weight of the veterinary composition. In particularly preferred embodiments, the carrier may be present in an amount of about 44% to about 62% by weight of the veterinary composition.

The thickening agent may be any excipient fit for purpose. For example, in preferred embodiments the thickening agent may be selected from the group consisting of one or more of silicone dioxide, castor wax, hydrogenated castor oil, paraffin and cetostearyl alcohol. In particularly preferred embodiments, the thickening agent includes silicon dioxide.

The thickening agent may be present in the veterinary composition in any amount fit for purpose. Preferably, the thickening agent is included in an amount which provides a smooth paste-like consistency. This has certain associated advantages: it may aid uniform distribution of the proton pump inhibitor in the veterinary composition and thereby prevent or reduce dosing inconsistencies, and; it may be generally difficult for an animal to reject upon oral administration. Thus, the thickening agent may be present in an amount of, for example, about 1% to about 15%, preferably about 1% to about 10%, by weight of the veterinary composition. In particularly preferred embodiments, the thickening agent may be present in an amount of about 3% to about 6% by weight of the veterinary composition.

It has been found to be an advantage of a veterinary composition of the present invention that an added preservative may not be necessary, especially in embodiments where the enteric coated form contains a preservative. Thus, while preservatives may be included, in preferred embodiments the veterinary composition does not contain an added preservative.

Veterinary compositions of the present invention are particularly useful for the prevention or treatment of a gastric ulcer condition in an animal. Thus, in another aspect, the present invention provides a method for the prevention or treatment of a gastric ulcer condition in an animal, said method including orally administering to said animal a veterinary composition as hereinbefore described. Preferably, the animal is a species selected from the group consisting of equine, porcine, bovine and canine, more preferably a horse or dog and most preferably a horse.

As used herein, by a “gastric ulcer condition” is meant gastrointestinal ulcers, including gastric or duodenal ulcers, or another gastrointestinal condition treatable using a proton pump inhibitor.

Administration of the veterinary composition may be directly into the mouth of an animal. For example, in a preferred embodiment the veterinary composition may be in the form of a paste dosage form in a dial-dose tube, and the end of the tube may be placed in the mouth and the veterinary composition expelled onto the back of the tongue of an animal.

The amount of the veterinary composition to be administered may vary according to a number of factors including, but not limited to, the species of animal to be treated, the type of dosage form, the proton pump inhibitor and its amount in the composition, the condition to be treated and its severity. A veterinarian skilled in the art may readily determine the amount of composition to be administered. For example, the amount of veterinary composition to be administered to an animal may be such that it delivers from about 0.01 mg of the proton pump inhibitor per kg of body weight to about 5 mg/kg of body weight per administration, preferably from about 0.05 mg/kg of body weight to about 2 mg/kg of body weight per administration, more preferably 0.1 mg/kg of body weight to about 1 mg/kg of body weight per administration. For example, for a 500 kg horse, the amount of proton pump inhibitor to be administered may be from about 5 mg to about 2500 mg, preferably from about 25 mg to about 1000 mg, more preferably from about 50 mg to about 500 mg.

The amount of proton pump inhibitor in the veterinary composition may determine the amount of composition to be delivered per administration, or vice versa. Preferably, the veterinary composition is formulated such that the amount of composition to be delivered per administration may be from about 1 mL to about 10 mL, more preferably about 2 mL to about 8 mL, or even more preferably about 2 mL to about 7 mL.

For treatment of a gastric ulcer condition, preferably the dosing regime consists of one to two administrations, preferably two administrations, per day for about 2 to about 4 weeks. This may be followed by a period of single administration per day for about 2 weeks, or for a period of time while the animal is in work or training or otherwise subject to, or predicted to be subject to, a stress event. For twice daily administration, preferably a first dose is given in the morning and a second dose at night. For once daily administration, preferably a dose is given in the morning. Preferably, the veterinary composition is administered before food. For prophylactic use aimed to substantially prevent a gastric ulcer condition, preferably the dosing regime consists of one to two administrations, preferably one administration, per day for about 2 to about 4 weeks or for a period of time while the animal is in work or training or otherwise subject to, or predicted to be subject to, a stress event. This may also include an initial loading dose prior to or during a stress event, which may be about 1.5 to about 3 times the usual dose.

By a “stress event” is herein intended to mean an event that may give rise to a gastric ulcer condition. This may include, for example, training, working, transportation, change to a feeding regime or environment, and/or pregnancy. Working may include racing.

When a single dose of the veterinary composition of the present invention is administered to an animal population, preferably a mean maximum plasma concentration (Cₘₐₓ) of proton pump inhibitor of greater than about 625 μg/L is reached. Independently, preferably a mean area under the curve (AUC) of plasma concentration of proton pump inhibitor over time of greater than about 1480 μg/L·hr is also reached. The dosage amount of the single dose may preferably be as hereinbefore described.

A person skilled in the art will appreciate that plasma concentrations and area under the curve values obtained in individual animals will vary due to variability in the many factors affecting drug absorption, distribution, metabolism and excretion. For this reason, mean values for an animal population are used herein. Specifically, given values for a mean pharmacokinetic parameter refer to a geometric mean unless otherwise indicated. The number of animals in an animal population is not particularly limited. For example, an animal population may include from a few to one hundred, several hundred or thousands of animals. In preferred embodiments the animal population includes at least 5, preferably at least 10, and more preferably at least 20, animals. For example, the animal population may include between 25 and 30 animals.

Thus, in another aspect, the present invention provides a veterinary composition for oral administration, said veterinary composition including a therapeutically effective amount of a proton pump inhibitor, wherein:
a mean maximum plasma concentration of proton pump inhibitor of greater than about 625 μg/L is reached in an animal population after administration to each animal a single dose of the veterinary composition; and/or
a mean area under the curve of plasma concentration of proton pump inhibitor over time of greater than about 1480 μg/L·hr is reached in an animal population after administration to each animal a single dose of the veterinary composition.

In preferred embodiments, the veterinary composition may be as hereinbefore described. For example, the veterinary composition may contain the proton pump inhibitor in an enteric coated form. The proton pump inhibitor may be a benzimidazole compound including omeprazole, lansoprazole, pantoprazole, rabeprazole and leminoprazole, preferably omeprazole. The veterinary composition may include an antacid, preferably magnesium oxide, and it may include a thickening agent and/or a carrier. The carrier may suspend or dissolve therein the other ingredients. The veterinary composition may be in a paste dosage form. Preferably, the animal is a species selected from the group consisting of equine, porcine, bovine and canine, and is most preferably a horse.

The single dose of the veterinary composition may be in an amount as hereinbefore described. For example, the single dose of the veterinary composition may be in an amount such that it delivers no more than about 5 mg proton pump inhibitor per kg of body weight of the animal. Preferably, the single dose of the veterinary composition may be in an amount such that it delivers from about 0.05 mg to about 2 mg, more preferably from about 0.1 mg to about 1 mg, of proton pump inhibitor per kg of body weight of the animal.

The mean maximum plasma concentration of proton pump inhibitor reached in the animal population by administration to each animal a single dose of the veterinary composition may be greater than about 650 μg/L, preferably about 700 μg/L or 750 μg/L, and more preferably about 800 μg/L or 850 μg/L.

For example, the mean maximum plasma concentration of proton pump inhibitor reached in the animal population may be between about 625 μg/L and about 1,000 μg/L, preferably between about 650 μg/L and about 1,000 μg/L, more preferably between about 700 μg/L or 750 μg/L and about 1,000 μg/L, and even more preferably between about 800 μg/L or 850 μg/L and about 1,000 μg/L.

The mean maximum plasma concentration may be reached in a mean time (Tₘₐₓ) no later than about 2, preferably about 1.5, and more preferably about 1.3, hours after administration of the veterinary composition. For example, the mean maximum plasma concentration may be reached on average between about 1 and about 2 hours after administration, preferably between about 1 and about 1.5 hours after administration, more preferably between about 1 and about 1.3 hours after administration of the veterinary composition.

The mean area under the curve of plasma concentration of proton pump inhibitor over time reached in the animal population by administration to each animal a single dose of the veterinary composition may be greater than about 1480 μg/L·hr, preferably about 1500 μg/L·hr or 1700 μg/L·hr, and more preferably about 1800 μg/L·hr or 1900 μg/L·hr.

For example, the mean area under the curve of plasma concentration of proton pump inhibitor over time reached in the animal population may be between about 1480 μg/L·hr and about 2100 μg/L·hr, preferably between about 1500, 1550, 1600 or 1650 μg/L·hr and about 2100 μg/L·hr, and more preferably between about 1700, 1750, 1800, 1850 or 1900 μg/L·hr and about 2100 μg/L·hr. Area under the curve may be measured by the trapezoidal approach to estimation taken from time zero to the last sampling time point at which proton pump inhibitor is quantifiable.

In preferred embodiments, both the mean maximum plasma concentration and mean area under the curve, as hereinbefore described, are reached in the animal population by administration to each animal a single dose of the veterinary composition. The dosage amount of the single dose may preferably be as hereinbefore described.

The veterinary composition of the invention may be manufactured by any suitable method. A person skilled in the art may readily determine a suitable manufacturing protocol. In general, a representative method for manufacture of, for example a paste dosage form, includes mixing the antacid with a carrier, adding with mixing a thickening agent then adding with mixing the enteric coated proton pump inhibitor.

Thus, in another aspect, the present invention provides a veterinary composition for oral administration, said veterinary composition including an antacid including magnesium oxide, a thickening agent, a carrier and a therapeutically effective amount of a proton pump inhibitor including omeprazole, wherein the proton pump inhibitor is provided in an enteric coated form, and the enteric coated form, antacid and thickening agent are suspended or dissolved in the carrier.

In preferred embodiments, the antacid, thickening agent, carrier, proton pump inhibitor, enteric coated form and amounts thereof in the veterinary composition may be as hereinbefore described. Also in preferred embodiments, when a single dose of the veterinary composition of the present invention is administered to an animal population, preferably a mean maximum plasma concentration of proton pump inhibitor of greater than about 625 μg/L is reached, as hereinbefore described. Independently, preferably a mean area under the curve of plasma concentration of proton pump inhibitor over time of greater than about 1480 μg/L·hr is also reached, as hereinbefore described.

The present invention will now be more fully described with reference to the accompanying Examples. It should be understood, however, that the description following is illustrative only and should not be taken in any way as a restriction on the generality of the invention described above.

### BRIEF DESCRIPTION OF THE FIGURES

FIG. 1 is a plot of mean omeprazole plasma concentration as a function of time for treatments using a test composition of the present invention (IVP) and a registered reference product (RVP) in a two-period cross-over design study testing efficacy in horses.

### DETAILED DESCRIPTION OF THE EMBODIMENTS

### Example 1—Formulation

A formulation of a veterinary composition of the present invention is exemplified in Table 1.

TABLE 1
Veterinary composition formulation.

| | Formulation | | | |
|---|---|---|---|---|
| | A | B | C | D |
| Ingredient | wt % in composition | | | |
| Magnesium oxide light | 15 | 13 | 13 | 13 |
| Omeprazole pellets | 21 | 31 | 37 | 39 |
| (omeprazole) | (4.2) | (9.3) | (3.1) | (3.2) |
| Canola oil | 61 | 50 | 47 | 44 |
| Silicon dioxide | 3 | 6 | 3 | 3 |

Composition A is formulated as a 50 mg/mL omeprazole composition using omeprazole pellets containing about 20 wt % omeprazole by weight of the pellets. Composition B is formulated as a 100 mg/mL omeprazole composition using omeprazole pellets containing about 30 wt % omeprazole by weight of the pellets. Compositions C and D are formulated as 40 mg/mL omeprazole compositions using omeprazole pellets containing about 8.3 wt % omeprazole by weight of the pellets. The omeprazole pellet formulation includes omeparazole on a sugar core with a hydroxypropylmethylcellulose sealing layer (i.e. HPMC-E5) and a methacrylic acid copolymer (i.e. L-30D) enteric coat.

Each composition is beige in colour with visible white enteric coated pellets distributed throughout. Compositions are formulated in a paste dosage form intended for administration to horses and packaged in 36 mL dial-dose tubes. Optimal viscosity as determined by USP 39<912> is in the range of about 4000 to about 12000 cP. Optimal pH as determined by USP 38<791> is in the range of about 10 to about 12. Optimal total impurity levels are less than 2 wt % as determined by USP 38.

Composition A containing 20 wt % omeprazole pellets is characterised by comparatively better consistency for packaging and administration, and uniformity of content for consistent dosage administration.

### Example 2—Manufacture

A veterinary composition of Example 1 is manufactured by the following process.
1. Set up the Planetary Mixer with the Paddle Mixer attachment.
2. Place the mixing vessel into place on the Planetary Mixer and lock the bowl into position. Add the canola oil to the mixing vessel.
3. Set the timer for fifteen (15) minutes and start the mixer on slow speed (Speed 1) and slowly add the Magnesium Oxide over two portions stopping the mixer intermittently so that the side of the bowl can be scraped.
4. Set the mixer (speed 1) and mix for a further five (5) minutes to ensure the Magnesium Oxide is dispersed and no lumps are present.
5. Set the timer for ten (10) minutes and add the Silicon Dioxide whilst continuously mixing for five (5) minutes on slow speed (speed 1). Stop the mixer (and timer) at the five (5) minute mark and scrape down the side of the vessel. Continue mixing for the remaining five (5) minutes on medium (speed 2). Once the ten (10) minutes total mixing time is complete, scrape down the side of the mixing bowl with a teflon scraper.
6. Add the Omeprazole Pellets over two portions and mix with intermittent mixing between each addition. Set the timer and perform a final mix of ten (10) minutes on medium (Speed 2) so that the Omeprazole pellets are evenly dispersed through the product.
7. Stop the mixer and timer and scrape the excess material from the vessel and paddle into the bowl at the five (5) minute mark. Mix thoroughly using the Teflon scraper.
8. Remove the bowl from the Planetary Mixer and cover the bowl with a HDPE bag and leave overnight before filling is commenced.

### Example 3—Stability Trials

Stability trials were performed on one (1) 42 Kg batch and two (2) supporting 6 Kg batches of a composition corresponding to formulation A of Example 1 packaged in dial-dose tubes. Table 2 presents the results of stability trials at storage conditions of 30±2° C. and 65±5% Relative Humidity (% RH) tube tip facing downwards for 3 months. Appearance and pack integrity were assessed by visual inspection. Standard techniques and parameters were used to determine viscosity (i.e. USP 39<912>), pH (i.e. USP 39<791>) impurities (i.e. USP 38) and assay.

TABLE 2
Storage 30 ± 2° C. and 65 ± 5% Relative Humidity (% RH) tube tip facing downwards.

| | | | | | Total | |
|---|---|---|---|---|---|---|
| Batch | | | | | impurities | Assay |
| No. | Appearance | Pack integrity | Viscosity cP | pH | wt % | mg/ml |
| 1 | Beige colour, white | Intact; no colour | 4900 | 11.0 | 0.45 | 51.8 |
| | pellets evenly distributed; | variation; no leaks | | | | |
| | no evidence of separation | | | | | |
| 2 | Beige colour, white | Intact; no colour | 5300 | 10.6 | 0.57 | 49.8 |
| | pellets evenly distributed; | variation; no leaks | | | | |
| | no evidence of separation | | | | | |
| 3 | Beige colour, white | Intact; no colour | 8400 | 10.0 | 0.42 | 48.9 |
| | pellets evenly distributed; | variation; no leaks | | | | |
| | no evidence of separation | | | | | |

Stability trials were also performed on a 50 Kg batch of a composition corresponding to formulation D of Example 1. Table 3 presents the results of accelerated stability trials at storage conditions of 40±2° C. and 75±5% Relative Humidity (% RH) inverted. Appearance was assessed by cutting the tube lengthways and without mixing visually assessing the colour and homogeneity of the composition. Pack integrity was assessed by visual assessment. SG, assay and micro data was determined using standard techniques and parameters. The micro data in particular demonstrates that it is not necessary to include an added preservative in a veterinary composition of the present invention.

TABLE 3
Storage 40 ± 2° C. and 75 ± 5% Relative Humidity (% RH) inverted.

| | | | | | Micro | | |
|---|---|---|---|---|---|---|---|
| Time | | | | Assay mg/ml, | Bacteria | Yeast/mould | |
| point | Appearance | Pack integrity | SG | % w/w (in pellets) | (cfug) | (cfug) | E. coli |
| 0 | Beige colour, white | Intact; no colour | 1.23 | 41.5, 8.0 | <10³ | <10² | Absent |
| | pellets evenly distributed; | variation; no leaks | | | | | |
| | no evidence of separation | | | | | | |
| +1 | Beige colour, white | Intact; no colour | 1.24 | 40.9, 8.2 | <10² | <10² | Absent |
| month | pellets evenly distributed; | variation; no leaks | | | | | |
| | no evidence of separation | | | | | | |
| +2 | Beige colour, white | Intact; no colour | 1.22 | 41.4, 8.9 | <10² | <10² | Absent |
| months | pellets evenly distributed; | variation; no leaks | | | | | |
| | no evidence of separation | | | | | | |
| +4 | Beige colour, white | Intact; no colour | 1.21 | 36.5, 8.2 | <10² | <10² | Absent |
| months | pellets evenly distributed; | variation; no leaks | | | | | |
| | no evidence of separation | | | | | | |

### Example 4—Experimental Trials

A sample population of 30 horses was treated with composition C or D of Example 1. Each horse was administered with a 5 mL dosage amount once daily for a period of at least 2 weeks or while in work or training. Symptoms of a gastric ulcer condition were absent from most to all horses after this treatment period. Veterinarian and Trainer feedback also included that horses treated with a veterinary composition of the present invention were characterised by the following:
Improved appetite;
Improved temperament;
Improved work/training performance;
Increased coat shine;
Decreased incidence of product rejection upon administration;
Increased willingness to receive treatment.

Reduced administration frequency was also reported in comparison with experiences with other products. For example, Veterinarians and Trainers noted that other products often require two to three administrations per day at higher dosage amounts for any observable effects, while a single administration of a veterinary composition of the present invention was sufficient in many cases for fast and effective treatment.

### Example 5—Efficacy Studies

A two-period cross-over design was used to test the efficacy in horses of a test composition of the present invention (IVP) compared with a market-leading registered reference product, marketed under the trade name Gastrozol (RVP). 28 horses were randomly separated into two even groups. Horses in one group received a single dose of the RVP in a first period and after a washout period then received a single dose of the IVP in a second period. Horses in the other group received a single dose of the IVP in a first period and after a washout period then received a single dose of the RVP in a second period. All dosage amounts were 5 mL.

The IVP was a composition A of Example 1 in paste dosage form delivering about 250 mg of omeprazole per 5 mL dose. The RVP was also in paste dosage form nominally formulated as a 50 mg/mL omeprazole composition delivering 250 mg omeprazole per 5 mL dose. The reverse-engineered formulation of the RVP is provided in Table 3.

TABLE 3
RVP formulation.

| Ingredient | approx. wt % in composition |
|---|---|
| Omeprazole beads | 31.3 |
| Paraffin oil | 68.3 |
| Diethyl phthalate; vanillin; | 0.4 |
| glycerol; parabens | |

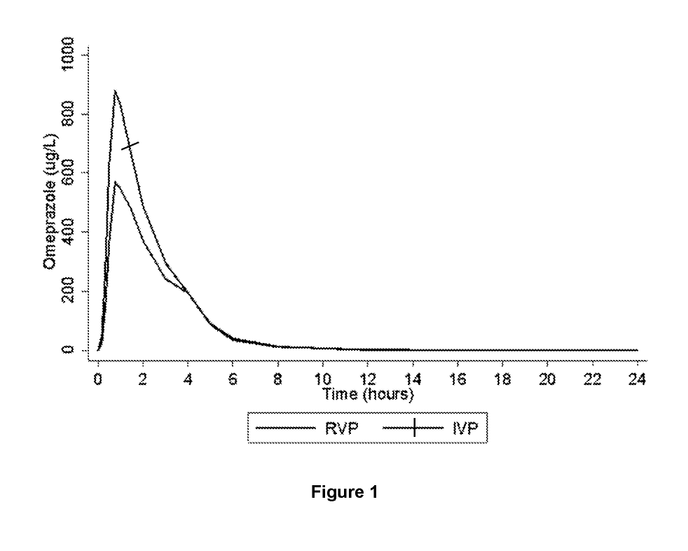

Plasma samples were taken from horses at a number of time intervals following dosage administration and analysed for omeprazole concentration. Data were assessed for pharmacokinetics parameters AUC, Cₘₐₓ and Tₘₐₓ. A plot of mean omeprazole plasma concentration as a function of time for IVP and RVP treatments is provided in FIG. 1.

Values for the measured pharmacokinetic parameters are provided in Table 4.

TABLE 4
Measured mean pharmacokinetic parameters.

| | AUC (μg/L · hr) | Cₘₐₓ(μg/L) | Tₘₐₓ(hr) |
|---|---|---|---|
| RVP | 1474.28 | 623.400 | 1.31 |
| IVP | 1922.31 | 886.884 | 1.17 |
| IVP − RVP | 448.03 | 263.484 | −0.14 |

The IVP achieved a statistically significant higher AUC compared to the RVP and a statistically significant higher Cₘₐₓ in less time (i.e. Tₘₐₓ).

It is to be understood that various alterations, modifications and/or additions may be made without departing from the spirit of the present invention as outlined herein.

As used herein, except where the context requires otherwise, the term “comprise” and variations of the term, such as “comprising”, “comprises” and “comprised”, are not intended to be in any way limiting or to exclude further additives, components, integers or steps.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and/or regarded as relevant by a person skilled in the art.

1. A veterinary composition for oral administration, said veterinary composition including an antacid and a therapeutically effective amount of a proton pump inhibitor, wherein the proton pump inhibitor is provided in an enteric coated form.

2. (canceled)

3. The veterinary composition according to claim 1, wherein the antacid is selected from one or more of the group consisting of: magnesium oxide, magnesium hydroxide, calcium carbonate, sodium bicarbonate, magnesium aluminate monohydrate, aluminium hydroxide, magnesium trisilicate, aluminium carbonate, magnesium carbonate, aluminium magnesium hydroxide sulfate and sodium citrate.

4. The veterinary composition according to claim 3, wherein the antacid includes magnesium oxide.

5. (canceled)

6. The veterinary composition according to claim 1, wherein the antacid is present in an amount of about 5% to about 25% by weight of the veterinary composition.

7. (canceled)

8. (canceled)

9. The veterinary composition according to claim 1, wherein the enteric coated form is present in an amount of about 5% to about 60% by weight of the veterinary composition.

10. (canceled)

11. (canceled)

12. (canceled)

13. (canceled)

14. (canceled)

15. (canceled)

16. (canceled)

17. (canceled)

18. The veterinary composition according to claim 1, wherein the proton pump inhibitor is selected from one or more of the group consisting of: omeprazole, lansoprazole, pantoprazole, rabeprazole and leminoprazole.

19. The veterinary composition according to claim 18, wherein the proton pump inhibitor includes omeprazole.

20. (canceled)

21. The veterinary composition according to claim 1, wherein the proton pump inhibitor is present in an amount of about 0.05% to about 55% by weight of the veterinary composition.

22. (canceled)

23. The veterinary composition according to claim 1, wherein the veterinary composition is in a paste dosage form.

24. The veterinary composition according to claim 1, wherein the veterinary composition includes a carrier selected from one or more of the group consisting of: mineral oil, avocado oil, castor oil, cottonseed oil, ethyl oleate, isopropyl myristate, isopropyl palmitate, myristyl alcohol, octyldodecanol, peanut oil, sunflower oil, capric triglycerides, caprylic triglycerides, safflower oil, sesame oil, canola oil, aniseed oil, macadamia nut oil, olive oil, squalene, rice bran oil, soya oil and almond oil.

25. (canceled)

26. The veterinary composition according to claim 24, wherein the carrier includes canola oil.

27. The veterinary composition according to claim 24, wherein the carrier is present in an amount of about 30% to about 85% by weight of the veterinary composition.

28. (canceled)

29. The veterinary composition according to claim 1, wherein the veterinary composition includes a thickening agent including silicon dioxide.

30. The veterinary composition according to claim 29, wherein the thickening agent is present in an amount of about 1% to about 15% by weight of the veterinary composition.

31. The veterinary composition according to claim 1, wherein the veterinary composition does not contain an added preservative.

32. A veterinary composition for oral administration, said veterinary composition including an antacid including magnesium oxide, a thickening agent, a carrier and a therapeutically effective amount of a proton pump inhibitor including omeprazole, wherein the proton pump inhibitor is provided in an enteric coated form and the enteric coated form, antacid and thickening agent are suspended or dissolved in the carrier.

33. The veterinary composition according to claim 32, wherein the veterinary composition is in a paste dosage form.

34. (canceled)

35. (canceled)

36. (canceled)

37. (canceled)

38. (canceled)

39. A method for the prevention or treatment of a gastric ulcer condition in an animal, including orally administering to said animal the veterinary composition according to claim 1.

40. The method according to claim 39, wherein the animal is a species selected from the group consisting of equine, porcine, bovine and canine.

41. The method according to claim 40, wherein the animal is a horse.

42. (canceled)
